(19) United States
(12) Patent Application Publication
(10) Pub. No.: US 2011/0236475 A1
(43) Pub. Date: Sep. 29, 2011

(54) GRANULAR PHARMACEUTICAL COMPOSITIONS

(75) Inventors: Mohammad Bala Pasha, Hyderabad (IN); Raghupathi Kandarapu, Hyderabad (IN); Vasanth Kumar Shetty, Hyderabad (IN); Indu Bhushan, Hyderabad (IN); Mailatur Sivaraman Mohan, Hyderabad (IN)

(73) Assignees: DR. REDDY'S LABORATORIES LTD., Hyderabad (IN); DR. REDDY'S LABORATORIES, INC., Bridgewater, NJ (US)

(21) Appl. No.: 13/157,621

(22) Filed: Jun. 10, 2011

Related U.S. Application Data
(63) Continuation of application No. 12/375,269, filed on Jan. 27, 2009.
(60) Provisional application No. 60/820,629, filed on Jul. 28, 2006.

(51) Int. Cl. A61K 9/48; A61K 9/50; A61K 31/445; A61K 9/20; A61P 37/08; B05D 5/00; B05D 7/00

(52) U.S. Cl. 424/452; 424/490; 514/317; 424/465; 427/2.1; 427/2.14

(57) ABSTRACT

Pharmaceutical compositions comprising a plurality of formulated particles containing at least one active ingredient and at least one pharmaceutically acceptable excipient, granulated with a granulating composition containing at least one pharmaceutical excipient.

The present invention relates to granular compositions comprising a plurality of formulated particles, processes to prepare the compositions, and optionally converting such granular compositions into finished dosage forms.

Various documents including U.S. Pat. Nos. 4,642,233, 5,643,602, 5,690,960, 5,753,265, 5,783,215, 5,910,319, and 6,136,344 disclose the preparation of pharmaceutical compositions such as tablets or capsules, comprising a plurality of formulated particles.

Some pharmaceutical formulations contain a plurality of formulated particles comprising an active ingredient, or an active ingredient physically blended with inert ingredients, or an inert plurality of formulated particles, filled into capsules, or the plurality of formulated particles are physically blended with pharmaceutically acceptable excipients or inert granules and such blends are compressed into tablets, as is known in processes for preparing formulations using a plurality of formulated particles. But such processes pose continuous challenges to the formulators, as there can be a large difference in physical properties between the plurality of formulated particles and the excipients or granules used. Size, shape and bulk density are a few physical properties that are critical and difficult to control. Frequently, formulated particles are comparatively more spherical in nature, whereas pharmaceutically acceptable excipients used in the processes together with a plurality of formulated particles are more irregular in shape. These differences in physical properties may result in problems such as loss of blend homogeneity, poor compressibility and surface rupture of the formulated particles, leading to processing issues and differences in content uniformity, release profiles and stability of formulations.

Hence, the development of compositions comprising a plurality of formulated particles comprising at least one active as described in the context of the present invention would be a significant improvement in the field of pharmaceutical technology.

### SUMMARY OF THE INVENTION

The present invention relates to granular compositions comprising a plurality of formulated particles, processes to prepare the compositions, and optionally converting such granular compositions into finished dosage forms.

An embodiment of the present invention provides granulated pharmaceutical compositions comprising a plurality of formulated particles and at least one pharmaceutically acceptable excipient, wherein a plurality of formulated particles contains at least one active ingredient and at least one pharmaceutically acceptable excipient.

In an embodiment, pharmaceutical compositions of the present invention exhibit a high degree of blend homogeneity with a relative standard deviation (“RSD”) less than about 6 of the mean assay value in a content uniformity determination.

In another embodiment of the present invention, a pharmaceutical composition comprises one or more active ingredients, wherein individual active ingredients are released immediately, or in a delayed or extended release manner, or in any combinations thereof.

In a further embodiment, the plurality of formulated particles of the present invention have an average particle size ranging from about 50 μm to about 5000 μm.

Another embodiment of the present invention provides a process for preparing a granulated pharmaceutical composition, comprising preparing a plurality of formulated particles containing at least one active ingredient and at least one pharmaceutically acceptable excipient, and granulating with a fluid containing at least one pharmaceutically acceptable excipient, and optionally converting said granulated pharmaceutical composition into a finished dosage form.

In an embodiment, a process to prepare said granulated pharmaceutical composition comprises wet granulation.

An aspect of the invention includes a pharmaceutical composition comprising a plurality of formulated particles containing at least one active ingredient and at least one pharmaceutically acceptable excipient, granulated with a granulating composition containing at least one pharmaceutical excipient.

Another aspect of the invention includes a process for preparing a pharmaceutical composition, comprising preparing a plurality of formulated particles containing at least one active ingredient and at least one pharmaceutically acceptable excipient, and granulating formulated particles with a granulating composition containing at least one pharmaceutically acceptable excipient.

A further aspect of the invention includes a pharmaceutical dosage form comprising a plurality of formulated particles containing at least one active ingredient and at least one pharmaceutically acceptable excipient, wherein a relative standard deviation of a mean weight of formulated particles presents in dosage form units is less than about 6 percent, from testing of ten units.

### DETAILED DESCRIPTION OF THE INVENTION

The present invention relates to granular compositions comprising a plurality of formulated particles, processes to prepare the compositions and optionally processes for converting such granular compositions into finished dosage forms.

“Active” is used herein synonymously with “active ingredient,” “active agent,” “pharmaceutical active agent” and “active pharmaceutical ingredient” and refers to a component of a composition that is present to provide a physiologic effect.

An aspect of the present invention provides granulated pharmaceutical compositions comprising a plurality of formulated particles comprising at least one active ingredient, granulated with a granulating composition comprising at least one pharmaceutically acceptable excipient.

In an embodiment, pharmaceutical compositions of the present invention exhibit a high degree of blend homogeneity with a relative standard deviation (“RSD”) less that about 6 percent of the mean assay value in a content uniformity test using ten determinations.

In another embodiment of the present invention, a pharmaceutical composition comprises one or more actives, wherein individual actives are released immediately, or in a delayed or extended release manner, or in combinations thereof.

The term “granulated pharmaceutical composition” or “co-granulate” in the context of the present invention relates to a granular blend comprising a plurality of formulated particles containing at least one active ingredient and at least one pharmaceutically acceptable excipient, that are co-processed using a granulation technique to obtain a granular blend having desired physico-chemical properties. Granulation is conducted using a granulating composition comprising at least one pharmaceutically acceptable excipient, and optionally at least one active ingredient. In certain embodiments, a single active ingredient can be present in both of a formulated particle and a granulating composition.

RSD is a widely used statistical term that indicates degree of variability from a mean of the data, and can be calculated using the following formula:

RSD (%)=100×(Standard deviation÷Mean).

In the context of the present invention, homogeneity has been measured in terms of content uniformity of the weight of formulated particles per finished dosage form unit. Typically, such determination of blend homogeneity involves dispersing a finished dosage form in a fluid where the formulated particles remain intact and undisturbed and can be separated from other excipients used in granulation of these particles and from any extra-granular excipients, separating the formulated particles, drying these particles and further determining the weight of dried particles from each finished dosage form, which is expressed as % w/w. The RSD can be calculated after determining the dried formulated particle weights in a number of dosage form units. A similar technique can be used to determine the homogeneity of blends comprising granulated compositions. Alternatively, the homogeneity can also be measured in terms of content uniformity in assay determinations, which involve determination of active content of various blend samples, and then statistically determining the mean value of assay along with RSD.

In an embodiment, granulated pharmaceutical compositions of the present invention comprise a plurality of formulated particles having an outer polymeric coating.

In another embodiment, a plurality of formulated particles of the present invention comprises pharmaceutically inert particulate cores, having a coating comprising at least one active ingredient.

In an embodiment of the present invention, at least one active is released from a plurality of formulated particles in an immediate or delayed or extended manner, or any combination thereof, or part of the active is released from formulated particles in an immediate or delayed or extended release manner, or any combination thereof, and other part is present outside the plurality of formulated particles in a granular portion to be released in an immediate or delayed or extended release manner, or any combination thereof.

In another embodiment, more than one actives are present in the compositions, wherein at least one active is released in an immediate or delayed or modified release manner, or any combination thereof, from a plurality of formulated particles, and at least one active is present in a different set of a plurality of formulated particles and is released in an immediate or delayed or modified release manner, or any combination thereof, or is present outside the plurality of formulated particles in a granular portion and is released in an immediate or delayed or modified manner, or any combination thereof.

In yet other embodiment more than one actives that ordinarily are incompatible with each other are present in different sets of pluralities of formulated particles, or one set of actives is present in plurality of formulated particles and another is outside the plurality of formulated particles in a granular portion, or in extra-granular excipients, optionally separated with inert coatings on one or more sets of formulated particles.

In a further embodiment, a finished dosage form comprises a plurality of formulated particles, granulated with a granulating composition comprising at least one pharmaceutically acceptable excipient and optionally at least one active, the granulate being blended with a composition comprising at least one active. This blend can be formulated into forms such as tablets and capsules.

The compositions comprise a plurality of formulated particles having average sizes ranging from 50-5000 μm, or from 100-2000 μm, or from 100-500 μm, or from 150-300 μm. These particles can be granules, spheroids, pellets, beads, seeds or cores. The cores typically are pharmacologically inert in nature and pharmaceutically compatible. Examples of various substances that can be used as cores include, but are not limited to: insoluble inert materials such as glass particles/beads or silicon dioxide, calcium phosphate dihydrate, dicalcium phosphate, calcium sulfate dihydrate, microcrystalline cellulose, cellulose derivatives, calcium carbonate, dibasic calcium phosphate anhydrous, dibasic calcium phosphate monohydrate, tribasic calcium phosphate, magnesium carbonate, magnesium oxide; soluble materials such as sugars like dextrose, lactose, anhydrous lactose, spray-dried lactose, lactose monohydrate, mannitol, starches, sorbitol, and sucrose; insoluble inert polymeric materials such as polyvinyl chloride, polystyrene or any other pharmaceutically acceptable insoluble synthetic polymeric material, and the like, and mixtures thereof.

A plurality of formulated particles of the present invention may comprise active alone, or active and at least one binder, or active and at least one pharmaceutically acceptable excipient from at least one class including diluents, disintegrants, binders, preservatives, anti oxidants, colorants, and the like; prepared using techniques such as granulation, extrusion-spheronization, powder layering, or solution or dispersion layering onto cores.

Co-granulates of the present invention may comprise one or more sets of pluralities of formulated particles, and at least one pharmaceutically acceptable excipient from at least one class including diluents, disintegrants, binders, preservatives, anti oxidants, colorants, and the like.

An embodiment of the present invention provides processes to prepare co-granulates comprising a plurality of formulated particles and at least one pharmaceutically acceptable excipient, and optionally converting said co-granulates into finished dosage forms.

In an embodiment, granular pharmaceutical compositions comprising a plurality of formulated particles are prepared by a wet granulation process comprising:
a) A plurality of formulated particles comprising at least one active together with at least one pharmaceutically acceptable excipient are formed by techniques such as powder coating, suspension or solution coating by any coating process, or fluidization using fluidized bed equipment, or extrusion and spheronization, and the like.
b) The plurality of formulated particles comprising at least one active are blended with pharmaceutically acceptable excipients.
c) The blend is granulated using a solvent or mixture of solvents, optionally with a binder or combination of binders.
d) The wet blend is dried.
e) The dried granular mass is sifted and optionally sized.
f) The granular mass is optionally lubricated and filled into sachets or bottles or capsules, or compressed into tablets and the tablets optionally coated with or without functional coating substances.

Granulation techniques that can be used in the present invention include but are not limited to wet granulation processes, such as fluid bed granulation processes. Low shear granulating equipment such as but not limited to a mass mixer, planetary mixer or fluid bed granulators are used to granulate the plurality of formulated particles along with other pharmaceutically acceptable excipients. Wet granulation techniques minimize physical stresses on the formulated particles, minimizing particle breakage.

In an embodiment, compositions of the present invention are prepared by a specific process comprising:
a) Dispersing or dissolving an active, with or without one or more pharmaceutically acceptable excipients such as binders, stabilizers, pH modifiers, anti oxidants, anti tacking agents and the like, in a solvent or a solvent mixture and spraying the dispersion or solution onto a plurality of inert particles in a fluid bed processor.
b) Dispersing or dissolving a release rate controlling substance in a solvent, with or without a plasticizer and other pharmaceutically acceptable excipients, and layering the dispersion or solution over an active-loaded plurality of formulated particles from step a), in a fluid bed processor.
c) Blending one or more pharmaceutically acceptable excipients or granules prepared from such excipients with a plurality of formulated particles from step b) and granulating the blend using a solvent or solvent mixture, with or without one or more pharmaceutically acceptable excipients such as binders, stabilizers, pH modifiers, anti oxidants, anti tacking agents and the like.
d) Drying the granular mass from step c), sifting and optionally sizing.
e) Optionally lubricating and filling into sachets or bottles or capsules, or compressing into tablets that can be uncoated or further coated as desired.

In another embodiment of the present invention, a plurality of formulated particles comprising active are prepared by:
a) blending active with one or more pharmaceutically acceptable excipients and processing through an extruder-spheronizer; or
b) layering the active as a powder onto inert beads or particles that are wetted with a solvent system optionally comprising binder; or
c) layering the active in the form of a suspension or solution with or without a binder in a fluid bed processor over inert beads or particles.

In another embodiment, a plurality of formulated particles comprising an active are used with or without film coating, or sugar coating, or coating with pH sensitive or pH independent release controlling substances.

In the present invention during the preparation of a plurality of formulated particles, or granulation of a plurality of formulated particles, or converting the granules into a finished dosage form, one or more pharmaceutically acceptable excipients may optionally be used. Useful pharmaceutically acceptable excipients include but are not limited to: diluents such as microcrystalline cellulose (MCC), silicified microcrystalline cellulose (“SMCC”, coprocessed 98% MCC and 2% colloidal silica and available from JRS Pharma of Rosenberg, Germany in various grades, e.g., Proslv™ HD 90 having an average particle size of 110 μm and a density of 0.25-0.37 g/cm³), microfine cellulose, lactose, starch, pregelatinized starch, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide and the like; binders such as acacia, guar gum, alginic acid, dextrin, maltodextrin, methylcellulose, ethyl cellulose, hydroxyethyl cellulose, hydroxypropyl cellulose (e.g. KLUCEL®), hydroxypropyl methylcellulose (e.g. METHOCEL®), carboxymethyl cellulose sodium, povidone (various grades of KOLLIDON®, PLASDONE®) starch and the like; disintegrants such as carboxymethyl cellulose sodium (e.g. Ac-Di-Sol®, Primellose®), crospovidone (e.g. Kollidon®, Polyplasdone®), povidone K-30, polacrilin potassium, starch, pregelatinized starch, sodium starch glycolate (e.g. Explotab®) and the like; surfactants including anionic surfactants such as chenodeoxycholic acid, 1-octanesulfonic acid sodium salt, sodium deoxycholate, glycodeoxycholic acid sodium salt, N-lauroylsarcosine sodium salt, lithium dodecyl sulfate, sodium cholate hydrate, sodium lauryl sulfate (SLS or SDS), cationic surfactants such as cetylpyridinium chloride monohydrate and hexadecyltrimethylammonium bromide, nonionic surfactants such as N-decanoyl-N-methylglucamine, octyl a-D-glucopyranoside, n-dodecyl b-D-maltoside (DDM), polyoxyethylene sorbitan esters like polysorbates and the like; plasticizers such as acetyltributyl citrate, phosphate esters, phthalate esters, amides, mineral oils, fatty acids and esters, glycerin, triacetin or sugars, fatty alcohols, polyethylene glycol, ethers of polyethylene glycol, fatty alcohols such as cetostearyl alcohol, cetyl alcohol, stearyl alcohol, oleyl alcohol, myristyl alcohol and the like. Solvents that are useful in layering or coating include but are not limited to: aqueous solvents such as water; organic volatile solvents such as acetaldehyde, acetone, benzene, carbon disulphide, carbon tetrachloride, 1,2 dichloroethane, dichloromethane, N,N-dimethylformamide, 1,4-dioxane, epichlorhydrin, ethyl acetate, ethanol, ethyl ether, ethylene glycol, 2-ethoxyethanol (acetate), formaldehyde, isopropanolol, methanol, methyl n-butyl ketone, methyl ethyl ketone, 2-methoxyethanol(acetate), perchloroethylene, toluene, 1,1,1-trichloroethane, trichloroethylene; and the like.

Pharmaceutical finished dosage forms of the present invention may further include other ingredients, such as but not limited to pharmaceutically acceptable glidants, lubricants, opacifiers, colorants, and other commonly used excipients.

The plurality of formulated particles comprising at least one active, or granules comprising a plurality of formulated particles, or finished dosage forms, can further be optionally film coated, or enteric coated, or seal coated, or coated with substances to modify the release of the active. The coating can be done by any techniques such as powder coating, spray coating, dip coating, fluidized bed coating and the like.

The release modifying and/or functional coating substances that can be used include but are not limited to: hydrophilic substances such as carboxymethyl cellulose sodium, hydroxyethyl cellulose, hydroxypropyl methylcellulose (HPMC); homopolymers or copolymers of N-vinylpyrrolidone; vinyl and acrylic polymers; polyacrylic acid and the like; hydrophobic substances such as celluloses like ethyl cellulose, low substituted hydroxypropyl cellulose (L-HPC), cellulose acetate, cellulose propionate (lower, medium or higher molecular weight), cellulose acetate propionate, cellulose acetate butyrate, cellulose acetate phthalate; polyalkyl methacrylates; polyalkyl acrylates; polyvinyl acetate (PVA); chitosan; crosslinked vinylpyrrolidone polymers; hydrogenated castor oil and the like. Other classes of rate controlling substances or their mixtures in various ratios as required are also within the purview of this invention without limitation.

Solvents used in the context of present invention in the processes of preparation of a plurality of formulated particles, or loading active onto a plurality of formulated particles, or granulating a plurality of formulated particles, or coating a plurality of formulated particles or granules comprising a plurality of formulated particles, or coating tablets, capsules, etc. prepared from granules comprising a plurality of formulated particles, include but are not limited to water, isopropyl alcohol, dichloromethane, acetone, ethanol, ethyl acetate, or combinations thereof in any ratio suitable for processing the compositions. Components in the solvent or solvent mixture may be present in solution or dispersion form in any ratio suitable for processing the compositions.

In context of the present invention, the active agents may include drugs or pharmaceuticals or nutraceuticals having therapeutic and/or nutritional value. The active can either be crystalline or amorphous in form, or mixtures thereof. Mixtures of actives are useful in the invention.

The pharmaceutical active agents comprise but are not limited to members of classes of actives including analgesics, anti-inflammatory agents, anthelmintics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, beta-blockers, cardiac ionotropic agents, corticosteroids, diuretics, anti-parkinsonian agents, gastro-intestinal agents, histamine receptor antagonists, keratolytics, lipid regulating agents, anti-anginal agents, cox-2-inhibitors, leukotriene inhibitors, macrolides, muscle relaxants, nutritional agents, opioid analgesics, protease inhibitors, sex hormones, stimulants, muscle relaxants, anti-osteoporosis agents anti-obesity agents, cognition enhancers, anti-urinary incontinence agents, nutritional oils, anti-benign prostate hypertrophy agents, essential fatty acids, non-essential fatty acids and the like.

Specific pharmaceutical active agents include but are not limited to: acetaminophen; acyclovir; acetyl cysteine; acetylcholine chloride; alatrofloxacin; alendronate; alglucerase; alfuzosin; amantadine hydrochloride; ambenomium; amifostine; amiloride hydrochloride; aminocaproic acid; amphotericin B; antihemophilic factor (human); antihemophilic factor (porcine); antihemophilic factor (recombinant); aprotinin; asparaginase; atenolol; atracurium besylate; atropine; azithromycin; aztreonam; BCG vaccine; bacitracin; becalermin; belladona; bepridil hydrochloride; bleomycin sulfate; calcitonin human; calcitonin salmon; carboplatin; capecitabine; capreomycin sulfate; cefamandole nafate; cefazolin sodium; cefepime hydrochloride; cefixime; cefonicid sodium; cefoperazone; cefotetan disodium; cefotoxime; cefoxitin sodium; ceftizoxime; ceftriaxone; cefuroxime axetil; cephalexin; cephapirin sodium; cholera vaccine; chorionic gonadotropin; cidofovir; cisplatin; cladribine; clidinium bromide; clindamycin and clindamycin derivatives; ciprofloxacin; clondronate; colistimethate sodium; colistin sulfate; cortocotropin; cosyntropin; cromalyn sodium; cytarabine; daltaperin sodium; danaproid; deforoxamine; denileukin diftitox; desmopressin; diatrizoate megluamine and diatrizoate sodium; dicyclomine; didanosine; dirithromycin; dopamine hydrochloride; dornase alpha; doxacurium chloride; doxorubicin; editronate disodium; elanaprilat; enkephalin; enoxacin; enoxaprin sodium; ephedrine; epinephrine; epoetin alpha; erythromycin; esmol hydrochloride; factor IX; famiciclovir; fludarabine; fluoxetine; foscarnet sodium; ganciclovir; granulocyte colony stimulating factor; granulocyte-macrophage stimulating factor; growth hormones-recombinant human; growth hormone-bovine; gentamycin; glucagon; glycopyrolate; gonadotropin releasing hormone and synthetic analogs thereof; GnRH; gonadorelin; grepafloxacin; hemophilus B conjugate vaccine; hepatitis A virus vaccine inactivated; hepatitis B virus vaccine inactivated; heparin sodium; indinavir sulfate; influenza virus vaccine; interleukin-2; interleukin-3; insulin-human; insulin lispro; insulin procine; insulin NPH; insulin aspart; insulin glargine; insulin detemir; interferon alpha; interferon beta; ipratropium bromide; isofosfamide; japanese encephalitis virus vaccine; lamivudine; leucovorin calcium; leuprolide acetate; levofloxacin; lincomycin and lincomycin derivatives; lobucavir; lomefloxacin; loracarbef; mannitol; measles virus vaccine; meningococcal vaccine; menotropins; mephenzolate bromide; mesalmine; mizolastine; methanamine; methotrexate; methscopolamine; metformin hydrochloride; metoprolol; mezocillin sodium; mivacurium chloride; mumps viral vaccine; nedocromil sodium; neostigmine bromide; neostigmine methyl sulfate; neutontin; norfloxacin; octreotide acetate; ofloxacin; olpadronate; oxytocin; pamidronate disodium; pancuronium bromide; paroxetine; pefloxacin; pentamindine isethionate; pentostatin; pentoxifylline; periciclovir; pentagastrin; phentolamine mesylate; phenylalanine; physostigmine salicylate; plague vaccine; piperacillin sodium; platelet derived growth factor-human; pneumococcal vaccine polyvalent; poliovirus vaccine inactivated; poliovirus vaccine live (OPV); polymixin B sulfate; pralidoxine chloride; pramlintide; pregabalin; propofenone; propenthaline bromide; pyridostigmine bromide; rabies vaccine; residronate; ribavarin; rimantadine hydrochloride; rotavirus vaccine; salmetrol xinafoate; sincalide; small pox vaccine; solatol; somatostatin; sparfloxacin; spectinomycin; stavudine; streptokinase; streptozocin; suxamethonium chloride; tacrine hydrochloride; terbutaline sulfate; thiopeta; ticarcillin; tiludronate; timolol; tissue type plasminogen activator; TNFR:Fc; TNK-tPA; trandolapril; trimetrexate gluconate; trospectinomycin; trovafloxacin; tubocurarine chloride; tumor necrosis factor; typhoid vaccine live; urea; urokinase; vancomycin; valaciclovir; valsartan; varicella virus vaccine live; vasopressin and vasopressin derivatives; vecoronium bromide; vinblastin; vincristine; vinorelbine; vitamin B12; warfarin sodium; yellow fever vaccine; zalcitabine; zanamavir; zolandronate; zidovudine; and pharmaceutically acceptable salts, isomers and derivatives thereof.

Useful pharmaceutical active agents further include but are not limited to aminoglutethimide, amiodarone, amlodipine, amphetamine, amphotericin B, atorvastatin, atovaquone, azithromycin, baclofen, beclomethasone, benezepril, benzonatate, betamethasone, bicalutanide, budesonide, bupropion, busulfan, butenafine, calcifediol, calcipotriene, calcitriol, camptothecin, candesartan, capsaicin, carbamezepine, carotenes, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clonazepam, clopidogrel, codeine, coenzyme Q10, cyclobenzaprine, cyclosporin, danazol, dantrolene, dexchlorpheniramine, diazepam, diclofenac, dicoumarol, digoxin, dehydroepiandrosterone, dihydroergotamine, dihydrotachysterol, dirithromycin, donezepil, efavirenz, eposartan, ergocalciferol, ergotamine, essential fatty acid sources, etodolac, etoposide, famotidine, fenofibrate, fentanyl, fexofenadine, finasteride, fluconazole, flurbiprofen, fluvastatin, fosphenytoin, frovatriptan, furazolidone, gabapentin, gemfibrozil, glibenclamide, glipizide, glyburide, glimepiride, griseofulvin, halofantrine, hydrochlorothiazide, ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide, loratadine, lorazepam, lovastatin, L-thryroxine, lutein, lycopene, medroxyprogesterone, mifepristone, mefloquine, megestrol acetate, methadone, methoxsalen, metronidazole, miconazole, midazolam, miglitol, minoxidil, mitoxantrone, montelukast, nabumetone, nalbuphine, naratriptan, nelfinavir, nifedipine, nilsolidipine, nilutanide, nitrofurantoin, nizatidine, omeprazole, oprevelkin, oestradiol, oxaprozin, paclitaxel, paracalcitol, paroxetine, pentazocine, pioglitazone, pizofetin, pravastatin, prednisolone, probucol, progesterone, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, terbutaline tetrahydrocannabinol, tiagabine, ticlopidine, tirofibran, tizanidine, topiramate, topotecan, toremifene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, zolmitriptan, zolpidem, zopiclone, and pharmaceutically acceptable salts, isomers and derivatives thereof.

Further, useful pharmaceutical active agents include cytokines, peptidomimetics, peptides, proteins, toxoids, serums, antibodies, vaccines, nucleosides, nucleotides, portions of genetic material, nucleic acids, and the like.

Useful nutraceuticals include but are not limited to: vitamins such as carotenoids, vitamin E, vitamin D, vitamin C, thiamine, riboflavin, niacin, folic acid, pyridoxine, biotin, pantothenic acid, cyanocobalamin and the like; minerals such as magnesium, manganese, zinc, selenium, chromium, copper and the like; and nutritional elements such as alpha lipoic acid, lutein, beta carotenoids, and the like.

The pharmaceutical compositions as disclosed in context of the present invention have uses including prophylaxis or treatment of diseases and disorders in mammals such as humans.

The following examples will further illustrate certain specific aspects and embodiments of the invention in greater detail and are not intended to limit the scope of the invention.

### EXAMPLE 1

Tablet composition comprising metoprolol sustained release coated pellets co-granulated with Prosolv.

| | | Grams/1000 |
|---|---|---|
| Ingredient | | Tablets |
| Drug- | SEAL-COATING | |
| Containing | Dicalcium phosphate anhydrous (A-Tab) * | 33 |
| Core | Ethyl cellulose 10 cPS | 4 |
| | Acetyltributyl citrate | 1 |
| | Isopropyl alcohol ‡ | 481 |
| | Methylene chloride ‡ | 241 |
| | DRUG-LOADING | |
| | Metoprolol succinate | 190 |
| | Hydroxypropyl methylcellulose (HPMC) | 22 |
| | Water ‡ | 495 |
| | Weight of drug-loaded pellet (mg) | 250 |
| Sustained | Ethyl cellulose | 120 |
| Release | Hydroxypropylmethyl cellulose (HPMC) | 26 |
| Coating | Acetyltributyl citrate | 29 |
| | Isopropyl alcohol ‡ | 1050 |
| | Methylene chloride ‡ | 525 |
| | Weight of SR coated pellet (A) (mg) | 425 |
| Granulation | Silicified MCC (Prosolv HD 90) ** | 416.27 |
| | Hydroxypropyl cellulose (Klucel LF) *** | 40.53 |
| | Water ‡ | 400 |
| Excipient | Hydroxypropyl cellulose (Klucel LF) *** | 30.00 |
| Blend | Croscarmellose sodium | 23.5 |
| for | Sodium stearyl fumarate | 4.7 |
| Tableting | Formulated tablet weight (B) (mg) | 940 |
| Film | Hydroxypropyl methylcellulose (HPMC) | 16.56 |
| Coating | Polyethylene glycol 6000 | 24.82 |
| | Talc | 2.06 |
| | Titanium dioxide | 16.56 |
| | Isopropyl alcohol ‡ | 760 |
| | Methylene chloride ‡ | 380 |
| | Film coating weight (C) (mg) | 60 |
| | Weight of finished tablet (A + B + C) (mg) | 1000 |

* A-Tab is dicalcium phosphate particles manufactured by Rhodia Inc., USA.
** Prosolv HD is manufactured by JRS Pharma GmbH Co. KG, Rosenberg, Germany.
*** Klucel LF is manufactured by Hercules Inc. and has a viscosity of 75-150 Pa · s (5% concentration in water).
‡ Evaporated during processing.

Manufacturing Process:
1. Dicalcium phosphate particles, ASTM mesh #80/#100, were seal-coated with a solution of ethyl cellulose and acetyl tributyl citrate dissolved in a mixture of isopropyl alcohol and methylene chloride, in a fluid-bed coater using a Wurster technique. After seal-coating, the sieve fraction of ASTM mesh #80/#100 was collected and further used for metoprolol succinate loading.
2. Metoprolol succinate was dissolved along with HPMC in water. Metoprolol succinate solution was sprayed over the seal-coated DCP core until the desired loading occurred, in a fluid-bed coater using Wurster technique.
3. Sustained release (SR) coating was prepared by dissolving ethyl cellulose and HPMC along with acetyl tributyl citrate in a mixture of isopropyl alcohol, methylene chloride, and water. SR coating of metoprolol succinate loaded pellets was carried out by Wurster technique using a fluid-bed coater with SR coating solution.
4. Co-granulation of SR coated pellets and Prosolv HD 90 was carried out in a fluid-bed coater using a top-spray technique. Klucel LF was dissolved in water and used as a binder solution. Both SR coated pellets and Prosolv HD 90 were charged into the fluid-bed coater bowl and granulation was carried out by the top-spray technique. The granulate was dried and sieved through a ASTM #20 sieve.
5. Granulated mass of step 4 was blended with Klucel LF, sodium stearyl fumarate, and croscarmellose sodium in a double-cone blender for 15 minutes.
6. Lubricated blend of step 5 was compressed into tablets using a 20-station rotary compression machine to get a hardness and weight of the tablets in the range of 8-12 kiloponds (“kP”) and 910-940 mg, respectively.
7. Core tablets were film-coated using a pan-coating technique. The film coating dispersion was HPMC, polyethylene glycol, talc, and titanium dioxide in a mixture of isopropyl alcohol and methylene chloride.

### Comparative Example A

Tablet composition of metoprolol SR coated pellets blended with Prosolv.

Ingredients were the same as in Example 1.

Manufacturing Process:
1. SR coated pellets (of Example 1, step 3) were blended with Prosolv HD 90 and Klucel LF (first quantity), then this blend was blended with Klucel LF (second quantity), croscarmellose sodium, and sodium stearyl fumarate in a double cone blender for 15 minutes.
2. Lubricated blend of step 1 was compressed into tablets using a 20-station rotary compression machine to get a hardness and weight of the tablets in the range of 8-12 kP and 910-940 mg, respectively.
3. Core tablets were film-coated using a pan-coating technique. Film coating solution was HPMC, polyethylene glycol, talc, and titanium dioxide in a mixture of isopropyl alcohol and methylene chloride.

### Comparative Example B

Tablet composition of metoprolol SR coated pellets blended with Prosolv granules.

Ingredients were the same as in Example 1.

Manufacturing Process:
1. Granulation of Prosolv HD 90 was carried out in a fluid-bed coater using Klucel LF dissolved in water, as a binder solution. Prosolv HD 90 was charged into fluid-bed coater top-spray bowl and granulation was carried out using a top-spray technique. After granulation and drying, granules of Prosolv HD 90 were passed through a ASTM #20 mesh sieve and used for blending and lubrication.
2. SR coated pellets (of Example 1, step 3) were blended with granulated Prosolv HD 90 mass of step 1 in a double-cone blender.
3. Klucel LF, sodium stearyl fumarate, and croscarmellose sodium were added to the step 2 mixture of SR coated pellets and granulated Prosolv HD 90, and blended for 15 minutes.
4. Lubricated blend of step 3 was compressed into tablets using a 20-station rotary compression machine to get a hardness and weight of the tablets in the range of 8-12 kP and 910-940 mg, respectively.
5. Core tablets were film-coated using a pan-coating technique. Film coating solution was HPMC, polyethylene glycol, talc, and titanium dioxide in a mixture of isopropyl alcohol and methylene chloride.

### EXAMPLE 2

Evaluation of blends.

Tablet blends were evaluated for particle size distribution by sieve analysis. Loss on drying was determined at 105° C. Also the blends were analyzed for bulk density and metoprolol content.

To determine the content uniformity, a finished tablet was dispersed in water and pellets from the dispersion were separated by passing through a ASTM #40 mesh sieve. The pellets retained on the sieve were dried, and the weight of dried pellets from each tablet was recorded and expressed as % w/w as a measure of homogeneity of the blend. Ten tablets were tested for the content uniformity RSD calculation.

| | | | % w/w | | |
|---|---|---|---|---|---|
| | | | Comparative | Comparative | |
| | | ASTM Mesh | Example A | Example B | Example |
| Testing | | Fraction | Blend | Blend | 1 Blend |
| Particle Size | SR Coated Pellets | #30/#50 | 90 | 90 | 90 |
| Distribution | Prosolv or its | #20/#40 | 0 | 5 | 0 |
| | Granules (Neat | #40/#60 | 2 | 35 | 2 |
| | Prosolv for | #60/#80 | 11 | 30 | 11 |
| | Example A and | #80/#100 | 17 | 20 | 17 |
| | Example 1, and | Below | 70 | 10 | 70 |
| | granules of step 1 | #100 | | | |
| | in Example B) | | | | |
| | Lubricated Blend | #30/#50 | 45 | 65 | 75 |
| | for Tableting | #50/#60 | 5 | 14 | 10 |
| | (products of step 1 | #60/#80 | 6 | 15 | 10 |
| | in Example A, step | Below | 44 | 6 | 5 |
| | 3 in Example B, | #100 | | | |
| | and step 5 in | | | | |
| | Example 1) | | | | |

| Bulk Density of Lubricated Blend | | 0.52 | 0.45 | 0.38 |
|---|---|---|---|---|
| for Tableting (g/ml) | | | | |
| Assay* of Lubricated Blend for | | 112.5 | 101.5 | 102 |
| Tableting | | | | |
| Content Uniformity | Mean | 105.6 | 103.2 | 105 |
| | Range | 78.9-125.6 | 88.4-117.5 | 98.3-112.1 |
| | RSD (%) | 14.6 | 8.6 | 4.4 |

*Pooled sample assay for metoprolol succinate (percent of theoretical amount).

Comparative Example A blend was heterogeneous when only Prosolv HD 90 was blended with the coated pellets. The Comparative Example B blend consisting of granulated Prosolv HD 90 and coated pellets resulted in homogeneity better than that of Comparative Example A blend with unprocessed Prosolv HD 90.

Co-granulation of coated pellets and Prosolv HD yielded the Example 1 blend with improved homogeneity. One of the reasons for improvement in the homogeneity of Example 1 blend with co-granulation of pellets and Prosolv HD 90 is arriving at a mixture having a narrow particle size distribution. Even though the bulk density of the Example 1 blend is lower, blend uniformity improves due to the narrow size distribution provided by the co-granulation process.

### EXAMPLE 3

Comparative in vitro dissolution profiles (n=6) of metoprolol. succinate SR tablets from Comparative Examples A and B, and Example 1.

Procedure: Test 711 “Dissolution” in United States Pharmacopeia 29, United States Pharmacopeial Convention, Inc., Rockville, Md., 2005.

Medium: Phosphate buffer pH 6.8

Apparatus: USP Apparatus II (rotating paddle)

Volume: 500 ml

Rotation speed: 50 rpm

“Mean” values are cumulative percent of drug dissolved.

| | Comparative | | Comparative | | | |
|---|---|---|---|---|---|---|
| Time | Example A | | Example B | | Example 1 | |
| (hours) | Mean | RSD* | Mean | RSD* | Mean | RSD* |

| | | | | | | |
|---|---|---|---|---|---|---|
| 0 | 0 | 0 | 0 | 0 | 0 | 0 |
| 1 | 31 | 12.3 | 18 | 8.5 | 14 | 4.5 |
| 4 | 47 | 8.5 | 36 | 7.6 | 33 | 3.8 |
| 8 | 69 | 6.8 | 56 | 4.6 | 48 | 3.1 |
| 20 | 102 | 2.3 | 98 | 2.1 | 96 | 1.9 |

*Relative standard deviation.

### EXAMPLE 4

Composition of fexofenadine hydrochloride 180 mg and pseudoephedrine hydrochloride 240 mg extended release tablets.

| | |
|---|---|
| Ingredient | Kg/5000 Tablets |

DRUG COATING

| | |
|---|---|
| Microcrystalline cellulose spheres | 0.1 |
| (Celphere CP-102)* | |
| Pseudoephedrine hydrochloride | 1.2 |
| Water‡ | 0.8 |
| Weight of drug-coated pellets (A) | 1.3 |

EXTENDED RELEASE COATING

| | |
|---|---|
| Ethyl Cellulose, 10 cPS | 1.2^{$} |
| Hydroxypropylmethyl cellulose (HPMC) | 0.3^{$} |
| Acetyltributyl citrate | 0.3^{$} |
| Isopropyl alcohol‡ | 10.9 |
| Methylene chloride‡ | 5.5 |
| Weight of ER coating (B) | 1.3 |

GRANULATION

| | |
|---|---|
| Fexofenadine hydrochloride | 0.9 |
| Silicified microcrystalline cellulose | 0.87 |
| (Prosolv HD 90) | |
| Copovidone (Plasdone S-630)# | 0.2 |
| Isopropyl alcohol‡ | 4.4 |
| Weight of intra-granular materials (C) | 2.3 |

LUBRICATION

| | |
|---|---|
| Silicified microcrystalline cellulose | 0.15 |
| (Prosolv HD 90) | |
| Croscarmellose sodium | 0.25 |
| Sodium stearyl fumarate | 0.03 |
| Weight of extra-granular materials (D) | 0.43 |

FILM COATING

| | |
|---|---|
| Hydroxypropyl methylcellulose, 5 cP | 0.09 |
| Polyethylene glycol 6000 | 0.15 |
| Talc | 0.02 |
| Titanium dioxide | 0.09 |
| Isopropyl alcohol‡ | 3.5 |
| Methylene chloride‡ | 3.5 |
| Weight of film coating (E) | 0.35 |

POLISHING

| | |
|---|---|
| Hydrogenated vegetable oil (Type I) | 0.1 |
| Total weight of tablets (A + B + C + D + E) | 5.36 |

^{$}Includes excess quantities to compensate for processing losses.
*Celphere CP-102 is manufactured by Asahi Kasei Chemical Corp., Japan.
#Plasdone S-630 is manufactured by International Specialty Products (ISP) Inc., New Jersey U.S.A.
‡Evaporated during processing.

Manufacturing Process:

A. Drug Coating
1. Pseudoephedrine hydrochloride was dissolved in water.
2. Solution of step 1 was coated onto microcrystalline cellulose spheres until a desired dose of the drug was built up, with a fluidized bed coater (FBC) using a Wurster technique.

B. Extended Release Coating
3. Ethyl cellulose and acetyltributyl citrate were dispersed in methylene chloride.
4. Drug coated microcrystalline cellulose spheres of step 2 were further coated with dispersion of step 3 with a fluidized bed coater (FBC) using a Wurster technique until a desired weight build-up was obtained.
5. The coated particles of step 4 were dried in the FBC at 60±5° C. for 2 hours.

C. Granulation
6. Fexofenadine hydrochloride and copovidone were dispersed in isopropyl alcohol.
7. Dried particles of step 5 were mixed with silicified microcrystalline cellulose, then granulated with the dispersion of step 6 in a fluidized bed processor using a top-spray technique.
8. The granules of step 7 were dried at 55±5° C. until the loss on drying (LOD) was less than 2% w/w, determined at 105° C.

D. Lubrication
9. Dried granules of step 8 were blended with a mixture of silicified microcrystalline cellulose, croscarmellose sodium and sodium stearyl fumarate in a double cone blender.

E. Compression
10. Lubricated blend of step 9 was compressed into tablets using a 21×10 mm punch set in a rotary compression machine to produce an average tablet weight of 1000 mg and tablet hardness in the range of 8-16 kP.

F. Film Coating
11. Hydroxypropyl methylcellulose, polyethylene glycol, talc and titanium dioxide were dispersed in a mixture of isopropyl alcohol and methylene chloride.
12. Tablets of step 10 were coated with dispersion of step 11 using a pan coating technique until a desired weight build-up was obtained.

G. Polishing
13. Coated tablets of step 12 were polished with hydrogenated vegetable oil using a pan coater by sprinkling vegetable oil over the warmed bed of tablets and tumbling for 30-45 minutes.

An antihistamine other than fexofenadine hydrochloride, such as loratadine, desloratadine, a cetirizine salt, a different fexofenadine salt, ebastine, mizolastine, etc., can also be used in the process to make tablets having similar therapeutic uses.

### Comparative Example C

Composition of fexofenadine hydrochloride 180 mg and pseudoephedrine hydrochloride 240 mg extended release tablets.

Prepared with the ingredients of Example 4, using a process similar to that described in Comparative Example A, which involves physical mixing of coated pellets with other excipients.

### Comparative Example D

Composition of fexofenadine hydrochloride 180 mg and pseudoephedrine hydrochloride 240 mg extended release tablets

Prepared with the ingredients of Example 4, using a process similar to that described in Comparative Example B, which involves blending of coated pellets with Prosolv granules.

Particle size distribution of lubricated blends for tableting:

% w/w Retained

| ASTM Mesh | Comparative Example D | Example 4 |
|---|---|---|
| No. | Lubricated Blend | Lubricated Blend |
| 35 | 41.0 | 56.6 |
| 40 | 4.4 | 3.8 |
| 60 | 21.1 | 5.9 |
| 80 | 15.5 | 10.3 |
| 100 | 4.7 | 7.5 |
| Below 100 | 13.4 | 15.8 |

Content uniformity was determined with ten tablets from each preparation, using a test method similar to that described in Example 2.

| | Comparative | Comparative | |
|---|---|---|---|
| Parameter | Example C | Example D | Example 4 |
| Mean | 108 | 102 | 100 |
| Range | 89-132 | 88-113 | 94-108 |
| RSD (%) | 13.9 | 8.7 | 4.9 |

### EXAMPLE 5

Preparation of metoprolol succinate extended release pellets.

| Ingredient | Grams/Batch |
|---|---|
| Dibasic calcium phosphate anhydrous (A-Tab) | 100 |

SEAL COATING

| | |
|---|---|
| Ethyl cellulose, 10 cP | 12 |
| Acetyltributyl citrate | 3 |
| Isopropyl alcohol ‡ | 180 |
| Dichloromethane ‡ | 95 |

METOPROLOL LAYER

| | |
|---|---|
| Seal coated pellets | 76 |
| Metoprolol succinate * | 380 |
| Hydroxypropyl methylcellulose 5 cP | 44 |
| Water ‡ | 788 |

EXTENDED RELEASE COATING

| | |
|---|---|
| Ethyl cellulose, 10 cP | 240 |
| Hypromellose 5 cP | 52 |
| Acetyltributyl citrate | 58 |
| Isopropyl alcohol ‡ | 4433 |
| Dichloromethane ‡ | 2217 |

* Amount expressed as the metoprolol tartrate equivalent.
‡ Evaporated during processing.

Manufacturing Process:
1. Ethyl cellulose and acetyltributyl citrate were dispersed in a mixture of isopropyl alcohol and methylene chloride.
2. The dispersion of step 1 was coated onto dibasic calcium phosphate using a fluidized bed coater (FBC) to produce a 15% weight gain.
3. Metoprolol succinate and hypromellose were dissolved in water to form a solution.
4. The drug solution of step 3 was coated onto seal coated cores of step 2 using a FBC to produce the desired weight gain. The particles were dried at 55±5° C. until the loss on drying (LOD) was less than 2% w/w, determined at 105° C.
5. Ethyl cellulose, hypromellose and acetyltributyl citrate were dispersed in a mixture of isopropyl alcohol and methylene chloride.
6. The ER coating solution of step 5 was coated onto drug loaded pellets of step 4 using a FBC to produce a 70% weight gain.

### EXAMPLE 6

Tablets containing metoprolol 100 mg in extended release form and hydrochlorothiazide 12.5 mg.

| | |
|---|---|
| Ingredient | Grams/500 Tablets |

GRANULATION

| | |
|---|---|
| Metoprolol succinate ER pellets (Example 5) | 106.3 |
| Prosolv HD 90 | 150 |
| Hydroxypropyl cellulose | 12.6* |
| Hydrochlorothiazide | 6.56* |
| Water‡ | 231.5* |

BLENDING AND LUBRICATION

| | |
|---|---|
| Hydroxypropyl cellulose | 4.7 |
| Croscarmellose sodium | 5.9 |
| Sodium stearyl fumarate | 1.2 |

*Contains 20% excess to account for processing losses.
‡Evaporated during processing.

Manufacturing Process:
1. Metoprolol succinate ER pellets of Example 5 were mixed with Prosolv.
2. Blend of step 1 was granulated with hydroxypropyl cellulose and hydrochlorothiazide suspension in water using a fluidized bed processor.
3. Granules of step 2 were blended with hydroxypropyl cellulose, croscarmellose sodium and sodium stearyl fumarate.
4. Lubricated blend of step 3 was compressed into tablets using a 11 mm round punch set to produce an average tablet weight of 350 mg.

### Comparative Example E

Tablets containing metoprolol 100 mg in extended release form and hydrochlorothiazide 12.5 mg.

Prepared using the Example 6 ingredients and a process similar to that described in Comparative Example A, which involves physical mixing of coated pellets with other excipients.

### Comparative Example F

Tablets containing metoprolol 100 mg in extended release form and hydrochlorothiazide 12.5 mg.

Prepared using the Example 6 ingredients and a process similar to that described in Comparative Example B, which involves blending of coated pellets with Prosolv granules.

Particle size distribution of lubricated blends for tableting:

| | % w/w Retained | | |
|---|---|---|---|
| | Comparative | Comparative | Example 6 |
| ASTM | Example E | Example F | Lubricated |
| Mesh No. | Lubricated Blend | Lubricated Blend | Blend |
| 35 | 11.0 | 16.0 | 48.4 |
| 40 | 35.3 | 42.5 | 12.9 |
| 60 | 4.9 | 22.2 | 19.4 |
| 80 | 4.5 | 6.3 | 6.5 |
| 100 | 4.1 | 1.9 | 1.2 |
| Below 100 | 40.2 | 11.0 | 11.7 |

Content uniformity was determined with ten tablets from each preparation, using a test method similar to that described in Example 2.

| | Comparative | Comparative | |
|---|---|---|---|
| Parameter | Example E | Example F | Example 6 |
| Mean | 103 | 109 | 107 |
| Range | 80-123 | 90-120 | 102-112 |
| RSD (%) | 13.9 | 8.6 | 3.6 |

### EXAMPLE 7

Enteric coated pellets of omeprazole magnesium.

| | |
|---|---|
| Ingredient | Grams/Batch |

SEAL COATING

| | |
|---|---|
| Sugar Spheres (#50/#60 mesh) | 1000 |
| Hydroxypropylmethyl cellulose 2910 | 70 |
| Water ‡ | 1330 |

DRUG LOADING

| | |
|---|---|
| Omeprazole magnesium | 123.81 |
| Hydroxypropylmethyl cellulose 2910 | 55 |
| Methanol ‡ | 507 |
| Dichloromethane ‡ | 507 |

SUB COATING

| | |
|---|---|
| Hydroxypropylmethyl cellulose 2910 | 70.0 |
| Talc | 120.0 |
| Magnesium stearate | 10.0 |
| Methanol ‡ | 570 |
| Dichloromethane ‡ | 570 |

ENTERIC COATING

| | |
|---|---|
| Methacrylic acid copolymer, type C (30% | 1025.4 |
| dispersion) | |
| Triethyl citrate | 38.44 |
| Talc | 40.43 |
| Titanium dioxide | 7.88 |
| Water ‡ | 859 |

‡ Evaporated during processing.

Manufacturing Process:
1. Hydroxypropyl methylcellulose (HPMC) 2910 was dissolved in water.
2. Sugar spheres were coated using the solution of step 1 to obtain a weight build-up of 5% w/w, in a fluidized bed processor with the following parameters:

| | | |
|---|---|---|
| Product temperature | 40-45° | C. |
| Atomization air pressure | 1.6-1.8 | bar |
| Spray rate | 4-6 | g/minute. |

3. HPMC 2910 was dissolved in a mixture of methanol and dichloromethane followed by addition of magnesium oxide and omeprazole magnesium.
4. Drug dispersion of step 3 was loaded on 300 g of seal coated sugar spheres from step 2 to obtain a weight gain of 53% w/w, using a fluidized bed processor with bottom spray and the following parameters:

| | | |
|---|---|---|
| Product temperature | 30-32° | C. |
| Atomization air pressure | 1.4 | bar |
| Spray rate | 12-16 | g/minute. |

5. HPMC 2910 was dissolved in a mixture of isopropyl alcohol and dichloromethane followed by addition of talc and magnesium stearate.
6. Drug loaded pellets (400 g) from step 4 were sub-coated with a dispersion of step 5 to a weight gain of 35% w/w, using a fluidized bed processor with bottom spray and the following parameters:

| | | |
|---|---|---|
| Product temperature | 30° | C. |
| Atomization air pressure | 1.6-1.8 | bar |
| Spray rate | 12-14 | g/minute. |

7. Methacrylic acid copolymer type C dispersion was dispersed in water.

Glyceryl monostearate (GMS) was dispersed in hot water and homogenized for 30 minutes and cooled to room temperature. Talc, titanium dioxide and triethyl citrate were added to the GMS dispersion and homogenized for 10 minutes. The GMS dispersion was mixed with polymer dispersion.
8. The subcoated pellets (200 g) from step 6 were enteric coated with the dispersion of step 7 to obtain a weight gain of 112% w/w, using a fluidized bed processor with bottom spray and the following parameters:

| | | |
|---|---|---|
| Product temperature | 28-30° | C. |
| Atomization air pressure | 1.8 | bar |
| Spray rate | 4-6 | g/minute. |

Other benzimidazole drugs can be used in place of omeprazole magnesium, including other salts of omeprazole and various salts of esomeprazole, lansoprazole, pantoprazole, rabeprazole, tenatoprazole, etc., to prepare compositions having similar therapeutic uses.

### EXAMPLE 8

Omeprazole magnesium tablets prepared by co-granulation of enteric-coated pellets from Example 7.

| | |
|---|---|
| Ingredient | Grams/Batch |

GRANULATION

| | |
|---|---|
| Enteric-coated pellets of Example 7 | 100 |
| Microcrystalline cellulose PH 302 | 148.17 |
| Hydroxypropyl cellulose (Klucel LF) | 14.51 |
| Water ‡ | 230 |

COMPRESSION

| | |
|---|---|
| Hydroxypropyl cellulose (Klucel LF) | 8.37 |
| Croscarmellose sodium | 6.7 |
| Sodium starch fumarate | 1.3 |

‡ Evaporated during processing

Manufacturing Process:
1. Hydroxy propyl cellulose was dissolved in water.
2. Enteric coated pellets of Example 7 and microcrystalline cellulose were co-granulated in a fluidized bed processor (top spray) with the following parameters:

| | | |
|---|---|---|
| Product temperature | 30° | C. |
| Atomization air pressure | 1.2 | bar |
| Spray rate | 4-6 | g/minute. |

3. Granules of step 2 were blended with hydroxypropyl cellulose and croscarmellose sodium for 10 minutes followed by blending with sodium starch fumarate for 5 minutes.
4. Lubricated blend of step 3 was compressed using 17.8 mm×6.8 mm caplet shaped standard concave dies and punches to a hardness of 8 kP with an average tablet weight of 750 mg.

### Comparative Example G

Omeprazole magnesium tablets prepared by physical mixing of enteric-coated pellets of Example 7 with other excipients.

Ingredients were the same as those of Example 8. Prepared using a process similar to that described in Comparative Example A, which involves physical mixing of coated pellets with other excipients.

Particle size distribution of lubricated blends for tableting:

% w/w Retained

| ASTM Mesh | Comparative Example G | Example 8 |
|---|---|---|
| No. | Lubricated Blend | Lubricated Blend |
| 35 | 28.3 | 18.7 |
| 40 | 12.6 | 21.0 |
| 60 | 22.2 | 4.5 |
| 80 | 19.3 | 5.2 |
| 100 | 7.9 | 6.2 |
| Below 100 | 9.7 | 44.4 |

Content uniformity was determined with ten tablets from each preparation, using a test method similar to that described in Example 2.

| Parameter | Comparative Example G | Example 8 |
|---|---|---|
| Mean | 107 | 103 |
| Range | 94-116 | 97-110 |
| RSD (%) | 7.4 | 4.3 |

1. A pharmaceutical composition comprising a plurality of formulated particles containing pseudoephedrine and at least one pharmaceutically acceptable excipient, the particles being granulated with a granulating composition containing fexofenadine and at least one pharmaceutical excipient.

2. The pharmaceutical composition of claim 1, wherein formulated particles comprise pharmaceutically inert particulate cores having a coating comprising pseudoephedrine.

3-8. (canceled)

9. The pharmaceutical composition of claim 1, wherein formulated particles are provided with an outer polymeric coating.

10. The pharmaceutical composition of claim 1, wherein formulated particles are provided with an outer coating that modifies release of pseudoephedrine.

11. (canceled)

12. The pharmaceutical composition of claim 1, wherein a portion of formulated particles releases pseudoephedrine in a manner that differs from the release of pseudoephedrine by another portion of formulated particles.

13. A pharmaceutical dosage form comprising the pharmaceutical composition of claim 1 and at least one pharmaceutically acceptable excipient, compressed into a tablet.

14. The pharmaceutical dosage form of claim 13, wherein a relative standard deviation of a mean weight of formulated particles present in dosage form units is less than about 6 percent, from testing of ten units.

15. A pharmaceutical dosage form comprising the pharmaceutical composition of claim 1, contained in a capsule.

16. (canceled)

17. The pharmaceutical dosage form of claim 15, wherein a relative standard deviation of a mean weight of formulated particles present in dosage form units is less than about 6 percent, from testing of ten units.

18. A process for preparing a pharmaceutical composition, comprising preparing a plurality of formulated particles containing pseudoephedrine and at least one pharmaceutically acceptable excipient, and granulating formulated particles with a granulating composition containing fexofenadine and at least one pharmaceutically acceptable excipient.

19. (canceled)

20. The process of claim 18, wherein a portion of formulated particles releases pseudoephedrine in a manner that differs from the release of pseudoephedrine by another portion of formulated particles.

21-22. (canceled)

23. The process of claim 18, wherein formulated particles are provided with an outer coating that modifies release of pseudoephedrine.

24. The process of claim 18, wherein formulated particles are provided with an outer polymeric coating.

25. (canceled)

26. The pharmaceutical composition of claim 1, wherein pseudoephedrine is present as a salt.

27. The pharmaceutical composition of claim 1, wherein pseudoephedrine is present as a hydrochloride salt.

28. The pharmaceutical composition of claim 1, wherein fexofenadine is present as a salt.

29. The pharmaceutical composition of claim 1, wherein fexofenadine is present as a hydrochloride salt.

30. A process for preparing a pharmaceutical composition, comprising:
a) providing pharmacologically inert particles with a coating comprising pseudoephedrine;
b) applying a coating comprising a polymer to particles from a); and
c) combining particles from b) with at least one pharmaceutical excipient and granulating with a fluid comprising an antihistamine.

31. The process of claim 30, further comprising:
d) combining granules from c) with at least one pharmaceutical excipient and compressing to form tablets.

32. The process of claim 30, further comprising:
d) combining granules from c) with at least one pharmaceutical excipient and filling into capsules.

33. The process of claim 30, wherein pharmacologically inert particles are microcrystalline cellulose.

34. The process of claim 30, wherein a coating in a) comprises a pseudoephedrine salt.

35. The process of claim 30, wherein a coating in a) comprises pseudoephedrine hydrochloride.

36. The process of claim 30, wherein an antihistamine comprises a fexofenadine salt.

37. The process of claim 30, wherein an antihistamine comprises fexofenadine hydrochloride.

38. A pharmaceutical composition, prepared by the process of claim 30.

39. The pharmaceutical composition of claim 38, being combined with at least one pharmaceutical excipient and made into a tablet or capsule dosage form.
